(19) United States
(12) Patent Application Publication
(10) Pub. No.: US 2023/0049313 A1
(43) Pub. Date: Feb. 16, 2023

(54) SYSTEM AND METHOD OF INTEGRATING TEXT MESSAGING READ NOTIFICATION WITH AN EMR SYSTEM

(71) Applicant: Teledact Inc., Thornhill (CA)

(72) Inventor: Tommy Tsz Him CHEUNG, Thornhill (CA)

(21) Appl. No.: 17/662,486

(22) Filed: May 9, 2022

Related U.S. Application Data
(60) Provisional application No. 63/232,424, filed on Aug. 12, 2021.
(60) Provisional application No. 63/232,427, filed on Aug. 12, 2021.

(51) Int. Cl. G16H 10/65; H04W 4/14

(52) U.S. Cl. CPC G16H 10/65; H04W 4/14

(57) ABSTRACT

A system and method for integrating a text messaging notification system with an electronic medical record (EMR) system. A messaging notification system that provides for secure text messaging to a mobile phone is integrated with an EMR system wherein the text messages are stored as medical records in the EMR system. No data is saved on the message notification system but stored directly in the EMR system. The EMR system is integrated with the mobile device operating system to support acknowledgement of read and delivery receipts.

### CROSS REFERENCE TO RELATED APPLICATIONS

The application claims priority to and the benefit of U.S. Utility Provisional Application Ser. No. 63/232,424, entitled “SYSTEM AND METHOD OF INTEGRATING TEXT MESSAGING READ NOTIFICATION WITH AN EMR SYSTEM”, filed on Aug. 12, 2021, and U.S. Utility Provisional Application Ser. No. 63/232,427, entitled “SYSTEM AND METHOD OF INTEGRATING TEXT MESSAGING NOTIFICATION FEATURES WITH AN EMR SYSTEM”, filed on Aug. 12, 2021, both of which are incorporated herein by reference in their entirety.

### BACKGROUND

The embodiments described herein relate to telemedicine, in particular, technologies related to integrations with EMR systems.

Electronic medical records (EMR) are used by doctors and clinician offices to store patient medical information including individual contact information, and patient medical treatment and history. Currently, some EMR systems, such as Oscar Pro EMR, do not have a built-in communication system, such as an instant messaging or text messaging system to communicate with patients. Further, these systems also lack the ability to securely store messages in each patient's medical chart.

It is time-consuming for staff at a clinic or doctor's office to call or email and subsequently manage the calls or emails for updates, missed appointments, rescheduling, and other administrative tasks. Furthermore, manually updating these interactions in the patient's medical record is labor-intensive. This typically requires logging into the software, searching for the patient chart, entering the update, saving, and logging out.

Furthermore, current short messaging system (SMS) systems do not support read receipt when sending SMS messages with existing EMR systems. When an SMS message is sent to a mobile phone, the system may not know if the user had read it or not.

With the Greater Toronto area being a multicultural city, there are multitudes of patients from different backgrounds and thus English can be a language barrier. A multilingual messaging tool would be highly useful. Some patients only have access to land-line telephones and this makes it difficult to constantly manage phone calls to them.

There is a further desire to provide read receipt or read notifications for sending SMS messages for EMR systems and updating the message record with read receipt in the EMR record with the intent to reduce the use of telephone calls to patients to inform them of administrative matters.

### SUMMARY

A system and method for integrating a text messaging notification system with an electronic medical record (EMR) system. A messaging notification system that provides for secure text messaging to a mobile phone is integrated with an EMR system wherein the text messages are stored as medical records in the EMR system. No data is saved on the message notification system, but stored directly in the EMR system. The EMR system is integrated with the mobile device operating system to support acknowledgement of read and delivery receipts.

### BRIEF DESCRIPTION OF THE DRAWINGS

FIG. 1 is a block diagram illustrating an exemplary EMR notification system.

FIG. 2 is a diagram illustrating the functionality of a messaging system with an EMR system.

FIGS. 3A and 3B are diagrams illustrating sending a messaging via a portal.

FIGS. 4A and 4B are diagrams illustrating receiving a message on a phone.

FIGS. 5A to 5D are diagrams illustrating review of a patient chart on an EMR system.

FIG. 6 is a diagram illustrating a further exemplary EMR notification system.

FIG. 7 is a diagram illustrating an exemplary Teledact notification system.

FIGS. 8A to 8F are diagrams illustrating an exemplary a mobile device detection workflow.

### DETAILED DESCRIPTION

According to the disclosure, electronic medical record (EMR) systems may not support mobile message or SMS messages. Current available communication method provided by the EMR between physician/clinic to patient does not provide read receipt/delivery confirmation. Furthermore, it is difficult and time consuming to deliver a message to the patient with acknowledgement and it is time consuming and costly to manually call patients for administrative reminders.

Commercial EMR system have limited application programming interface (API) access whereby database structure may be fixed and does not allow any third party vendor to access, change or update records. Further, commercial EMR systems and open source EMR systems may not support mobile message with read receipt acknowledgement capabilities. In a further embodiment, SMS communication will reduce missed appointments and return the lost hours (from calling/email patients) back to clinic staff with the ability to quickly communicate with patients via SMS messages. The messages sent to patients are automatically recorded in an EMR system (i.e., Oscar Pro) medical chart without the need for staff to manually update them.

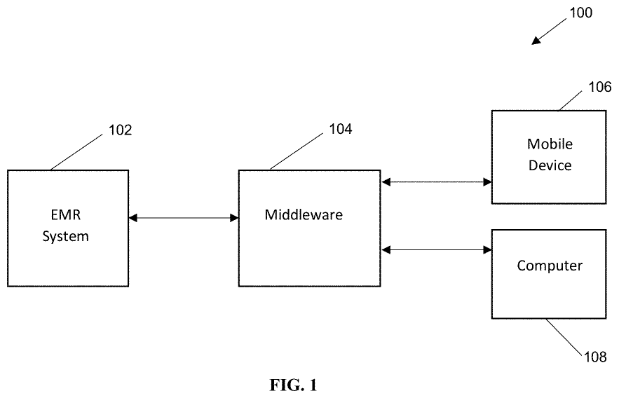

FIG. 1 is a block diagram illustrating an exemplary EMR notification system. According to FIG. 1, EMR notification system 100 consists of an EMR system 102 such as Oscar Pro used to store patient electronic medical records (EMR). A software middleware layer 104 connects to the EMR system 102 and also connects to one or more mobile device 106 or computer 108 to provide EMR notification messages. The notification messages can be email, SMS text messages or voice calls.

According to FIG. 1, the software middleware layer 104 includes application programming interface (API) connections to different messaging protocols, that enable the EMR system 102 to interface to applications on the mobile device 106 and computer 108. According to further aspects of FIG. 1, the software middleware layer 104 will be a tool that enables different stakeholders to communicate. For example, the middleware layer 104 will enable patient to communicate with a coordinator, a coordinator to communicate with a pharmacy, and a coordinator to communicate with a physician.

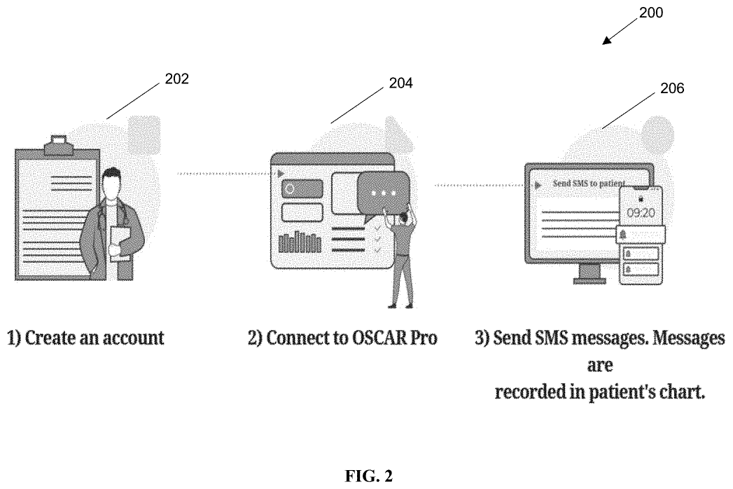

FIG. 2 is a diagram illustrating the functionality of a messaging system with an EMR system. According to FIG. 2, users of the clinic or doctor's office would create a patient account at step 202 in the EMR system 200. The user would then connect to the EMR system such as Oscar Pro at step 204. Thereafter, the user would send a short message service (SMS) text message to the patient's phone at step 206. The SMS message will be recorded in the patient's chart or record at step 206 within the EMR system 206.

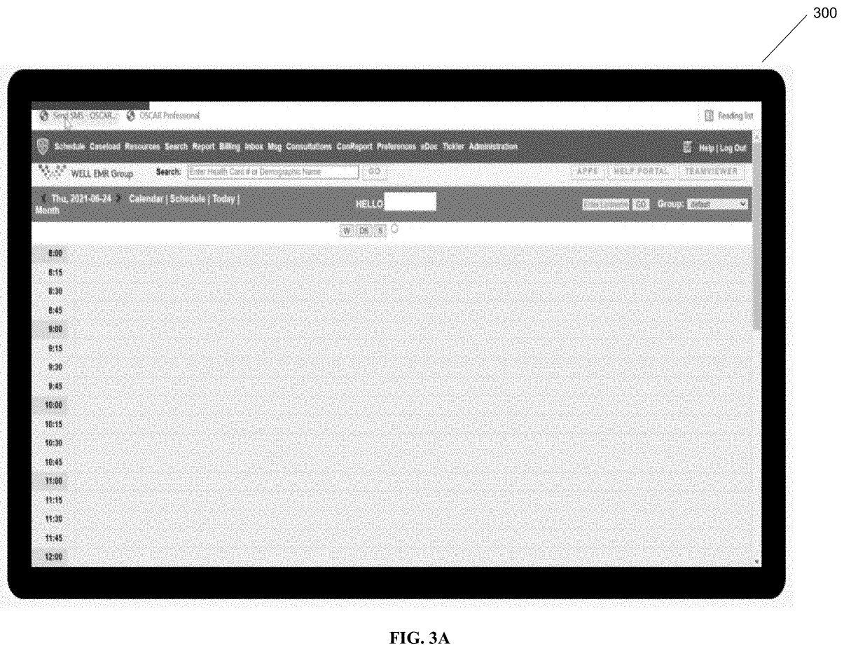

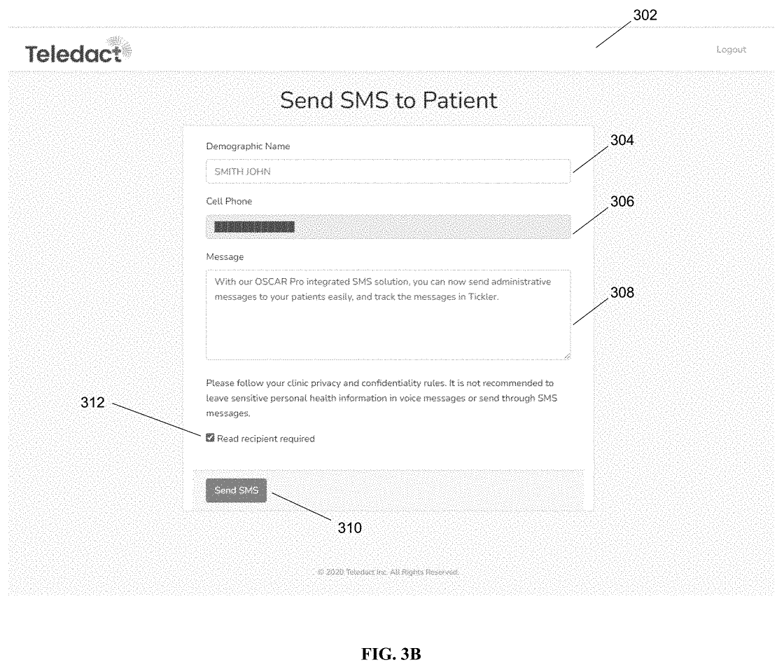

FIGS. 3A and 3B are diagrams illustrating sending a messaging via a portal. According to FIG. 3A, a messaging portal 300 is shown. A user or staff would log onto the EMR system and select the selection “Send SMS”. According to FIG. 3B, a Send SMS to Patient user interface 302 is shown. A message is sent from user or staff to patient from a portal page 302 where the staff enters patient name 304, mobile phone number 306 (e.g., cell phone field) and then type out a message 308. Thereafter, the message is sent to the patient (recipient) once the Send SMS button is selected. A further option box 312 to request read receipt is also provided which will allow the system to provide notification whether the message is read if this feature is supported (i.e., for mobile devices). In further embodiments, the mobile phone number field 306 may be replaced with a landline telephone number, email, voicemail, instant message or translation service.

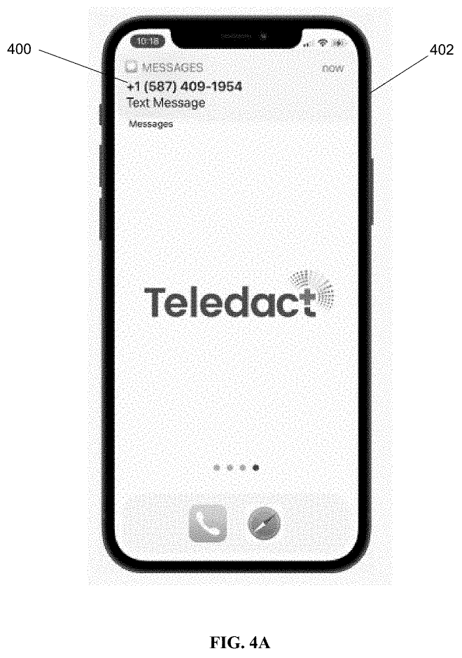

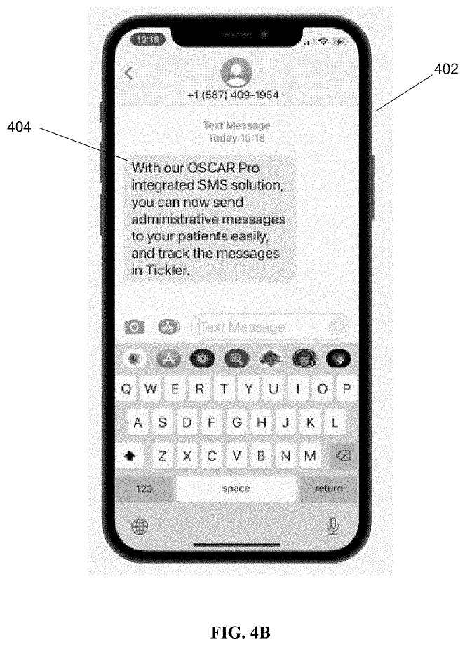

FIGS. 4A and 4B are diagrams illustrating receiving a message on a mobile phone. FIG. 4A, shows a text message (i.e., SMS message) notification 400 on a mobile phone 402. FIG. 4B illustrates the text message 404 received on the mobile device. In addition to received SMS message on a mobile phone, the message can also alternatively be sent to an email, an instant message service, a landline phone or to voice mailbox.

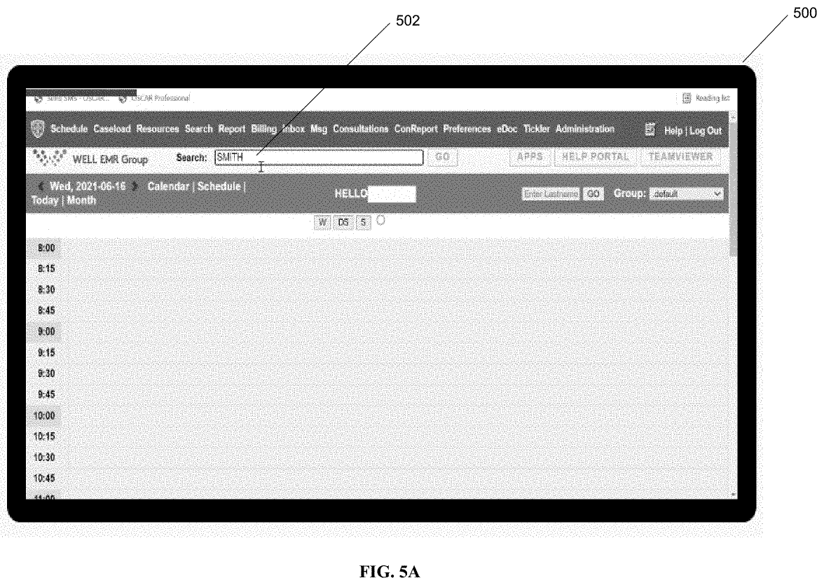

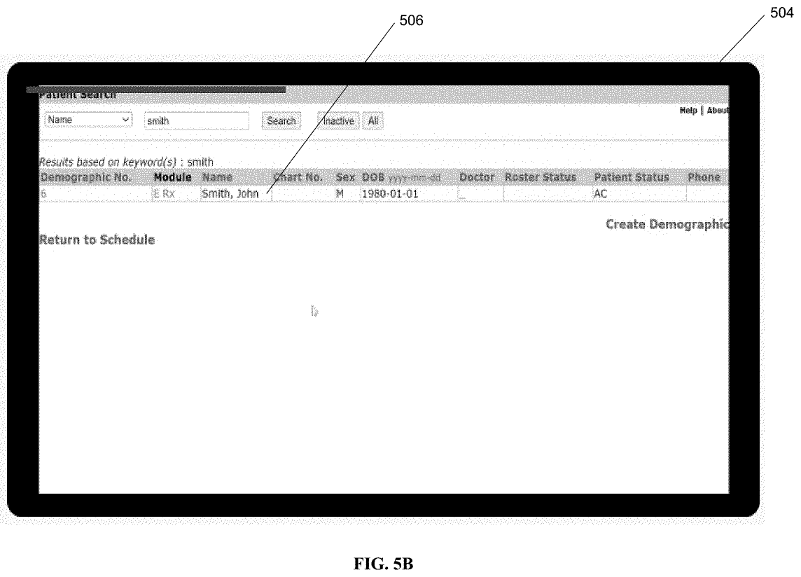

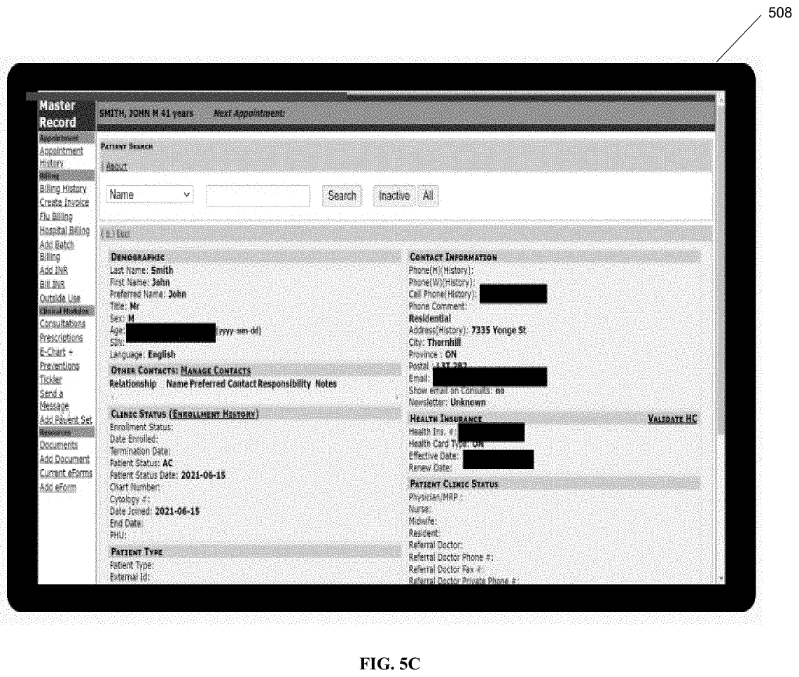

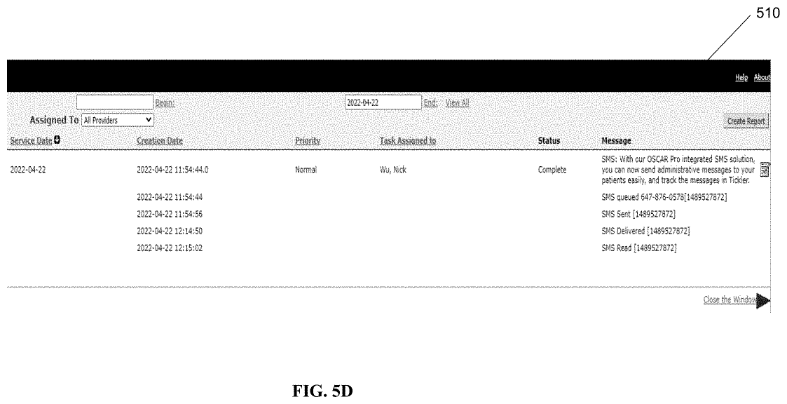

FIGS. 5A to 5D are diagrams illustrating review of a patient chart on an EMR system. FIG. 5A illustrates exemplary dashboard 500 of an EMR system. The user types in a patient, for example “Smith” in the Search bar 502. FIG. 5B is a diagram illustrating the search results for “Smith” 504. As shown in FIG. 5B, one record for “John Smith” 506 is retrieved. FIG. 5C illustrates the master record for the patient “John Smith” 508. FIG. 5D is a further screenshot of the master record illustrating recorded SMS messages 510. As seen in FIG. 5D, the SMS message sent to the mobile phone (FIG. 4A and FIG. 4B) are stored within the EMR system and can be retrieved and displayed to the user. Text and email messages are automatically (and permanently) recorded in the patient's medical chart.

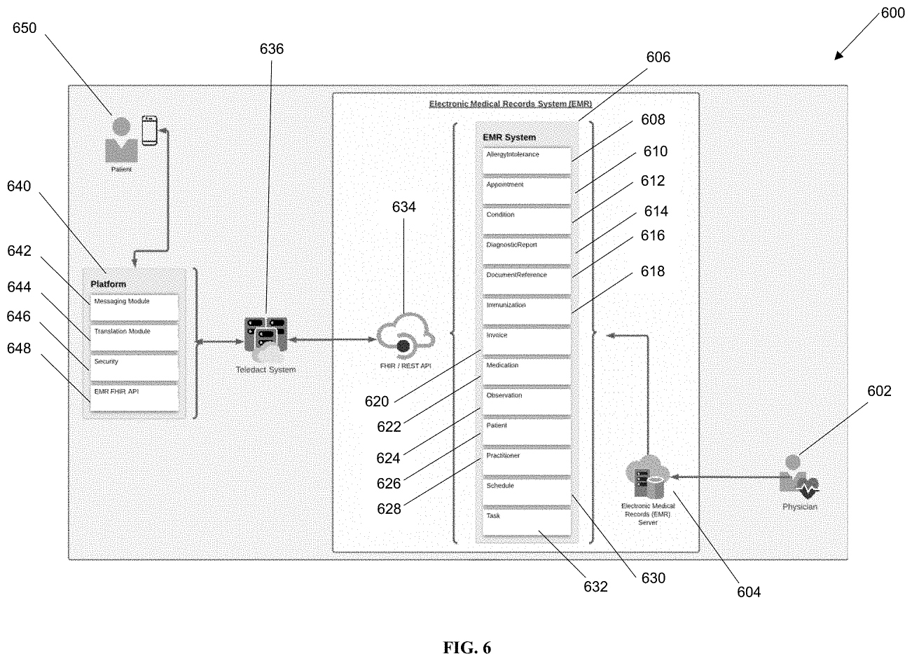

FIG. 6 is a diagram illustrating a further exemplary EMR notification system. According to FIG. 6, EMR notification system 600 consists of a message created from a physician or clinic 602 sent to the Electronic Medical Records (EMR) server 604 which is then routed to the EMR system 606.

EMR system 606 consists of a plurality of components or modules including an AllergyIntolerance module 606, an Appointment module 608, a Condition module 610, a DiagnosticReport module 612, a DocumentReference module 614, an Immunization module 616, an Invoice module 618, a Medication module 622, an Observation module 624, a Patient module 626, a Practitioner module 628, a Schedule module 630 and a Task module 632.

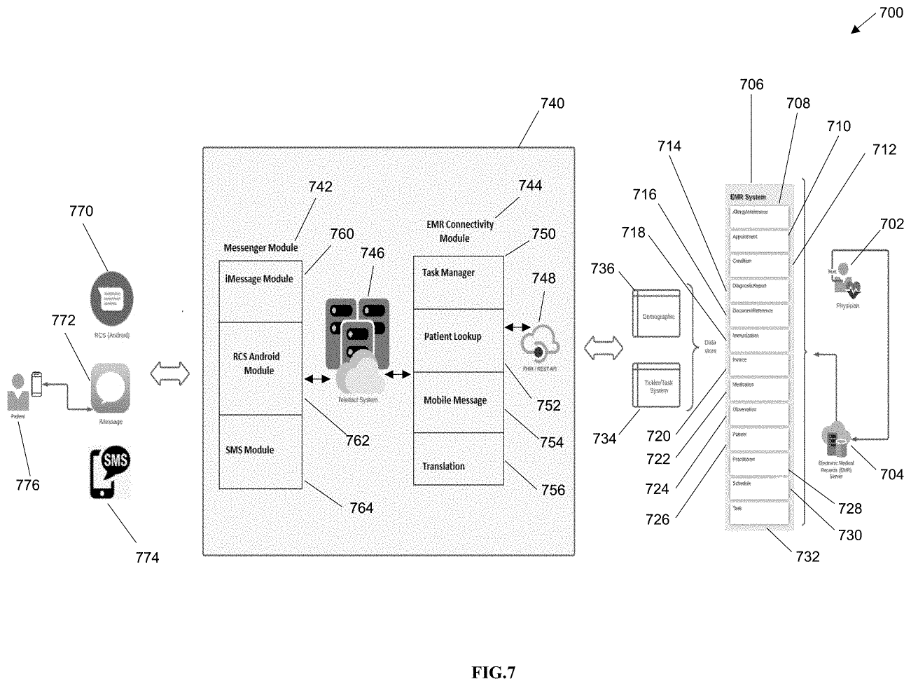

According to FIG. 6, the components or modules of EMR System 606 connect to a FIHR API or REST API 634 to the Teledact System 636. The FIHR API (Fast Healthcare Interoperability Resources Application Programming Interface) is a standard describing data formats and elements and an application programming interface for exchanging electronic health records. The standard was created by the Health Level Seven (HL7) International health-care standards organization. The REST API (Representational State Transfer Application Programming Interface) is a software architectural style that was created to guide the design and development of the architecture for the World Wide Web. REST defines a set of constraints for how the architecture of an Internet-scale distributed hypermedia system, such as the Web, should behave. More details on the Teledact System 636 will be elaborated in FIG. 7.

According to FIG. 6, Teledact System 636 connects to one or more Platform 640 whereby the message is then sent to the recipient or Patient 650. Platform 640 consists of Messaging Module 642, Translation Module 644, Security Module 646 and EMR FHIR API Module 648.

According to the disclosure, Translation Module 644 enables Teledact System 636 multi-lingual localization capabilities. Messages created from the EMR portal, in English, can be received by patients as a bilingual text or voice message (English and 1 other language). There is also an option for patients to choose one language of their choice (i.e., French, German, Dutch, Spanish, Mandarin Chinese, Cantonese Chinese, Punjabi, Japanese, Vietnamese, etc). Once the message is entered and stored in English text, it can be sent to Translation Module 644 whereupon it can be machine translated into another text message and/or verbally recorded into a voice message in a second language, whereupon it can be delivered in the second language via SMS text message, email message or “.wav” file as a voice note.

FIG. 7 is a diagram illustrating an exemplary Teledact notification system. According to FIG. 7, Teledact notification system 700 consists of a message created from a physician or clinic 702 sent to the Electronic Medical Records (EMR) server 704 which is then routed to the EMR system 706.

EMR system 706 consists of a plurality of components or modules including an AllergyIntolerance module 708, an Appointment module 710, a Condition module 712, a DiagnosticReport module 714, a DocumentReference module 716, an Immunization module 718, an Invoice module 720, a Medication module 722, an Observation module 724, a Patient module 726, a Practitioner module 728, a Schedule Module 730 and a Task module 732.

Records from the notification 700 are then saved in a data store which is connected to a Demographic module 736 and a Tickler/Task system module 734.

According to FIG. 7, EMR System 706 connects to Teledact System 740. Teledact System 740 further consists of Message Modules 742, Teledact servers 746 and EMR Connectivity Module 744. EMR Connectivity Module 744 consists of Task Manager module 750, Patient Lookup module 752, Mobile Message module 754 and Translation module 756.

According to the disclosure, the Task Manager module 750 has the following features:
System has its own custom algorithm and logic conditions to detect if a task is required to send a mobile messenger to the patient. For example, if the task is assigned to a Teledact system defined user account, then Teledact will retrieve these tasks and send it as a mobile message and update the EMR with delivery and read receipt.
System checks the EMR to detect if there is a task matching the Teledact system's defined logic and send automated mobile message and update EMR with delivery and read message status.
Physicians or clinic operators can create a task and assign to Teledact user account, and Teledact system will auto retrieve this task and process it to send as mobile messenger to the patient with delivery and read receipt.
With this automation and custom logic, this task can be marked completed automatically if system detects a read receipt from the patient's mobile messenger response.

According to the disclosure, the Patient Lookup module 752 has the following features:
System can search EMR demographics with patient info, not limited to name, address, phone numbers, cell phone, email, phone type.
System can update EMR demographics with patient info including phone type.

According to the disclosure, the Mobile Message module 754 has the following features:
System able to utilize RCS Android® module to detect if the phone is RCS enabled. If phone is RCS enabled, set phone type=RCS—Android®, continue to send RCS message and update EMR and System with delivery and read message status. If phone is not RCS enabled, try sending message via iMessage® bridge and obtain delivery & read receipt. If sent successful, set phone type=iMessage®. If sent unsuccessful, set phone type=SMS and continue to send message as SMS with 2 way communication to detect for read receipt.
System can also send a web uniform resource locator (URL) link to access the encrypted message, system will prompt the patient to enter a secret number that system sent to the mobile phone via the messenger module. User will need to enter the correct secret number in order to access and decrypt this message. This process will detect the phone type and update the EMR and system.

Messenger Module 742 further consists of iMessage® Module 760, RCS Android® Module 762 and SMS Mobile Module 764. According to the disclosure, the iMessage® Module 760 has the following:
Module to detect if phone is iMessage® enabled or not.
Bridge to send iMessage® with delivery and read receipt support.
Algorithm to identify phone type/message support stack for the phone number.

According to the disclosure, the RCS Android® Module 762 has the following features:
Module to detect if phone is RCS enabled—single or bulk lookup.
Send message to Android® RCS enabled device with delivery and read receipt.
Algorithm to identify phone type/message support stack for the phone number.

According to the disclosure, the RCS Android® Module 762 has the following features:
Module to send SMS.
Algorithm to identify phone type/message support stack for the given phone number.
Support 2-way communications with checkpoints logic.
Example 1: Send Notification VIA SMS “clinic message. Please Reply 1 to confirm received the message. System update read receipt.”
Example 2: Send message to patient's phone, provide URL link for response, system detect mobile device type when user click on the link to view the secure message and update EMR and system with phone type.

According to FIG. 7, Teledact System 740 further comprises one or more Teledact computer servers 746 that is either hosted on the cloud or at Teledact's office. Teledact computer server 746 provides the following functions:
Auto detect if there is a new task in EMR that needs to send automated mobile message. For example, if task is assigned to auto messenger, then system will retrieve this task and send automated messenger w/delivery & read receipt, update message status in EMR.
Lookup EMR with patient info, phone type and cell phone number.
Lookup Teledact System with phone type.
Update EMR Message/Task/Tickler with delivery receipt and read receipt.
Module to send mobile message via the messenger module (iMessage® bridge, Android® RCS Module, or SMS Module) based on the phone device type detected from our algorithm.
Module to send mobile message with web URL link to read encrypted message. When patient open the web URL link to access the encrypted message, system will prompt the patient to enter a secret number that sent to their phone in order to decrypt the secure message, at the same time the system will auto send a random generated secret number to the phone number via mobile messenger, when user enter this secret number to the online system, a decrypted secure message will be display. During this process, system will record and update the EMR and system this phone type.

According to FIG. 7, Teledact System 740 further connects to a mobile device and will send a message to RCS Android® 742, iMessage® 772 and/or SMS 774 which will be routed the mobile device of the Patient 776.

### Read Receipts

In further embodiments of this disclosure, the EMR system may also provide support for the ability to send messages to any users and able to get confirmation of read receipt and update from the EMR system accordingly. As an example, the EMR system will be able to send SMS messages to any mobile phone and once the patient has read the message, the system will know they had read the message and update the EMR system so that the clinic staff or physicians have confirmation they read the notification/message.

It is important to be able to track the read receipt for important message. In further embodiments, if the user's mobile phone does not support this read receipt acknowledgement, then system will send a link to click and confirm read for this important message/notification. The read receipts will be stored in the personal history of the patient with relevant fields such as date/time message sent and date/time message read.

Delivery of read receipts to the EMR system relies on integration with mobile operating systems such as Android® and OS (e.g., SDK or API calls in Android® and iOS to receive read receipts) and mobile device support for Rich Communication Services (RCS). In further embodiments, read receipt may also be configured in SIM toolkit settings.

In further embodiments, in addition to delivering read receipt, the EMR system may also provide support for delivery receipts which indicates whether a message has been successfully delivered to the receiver's device.

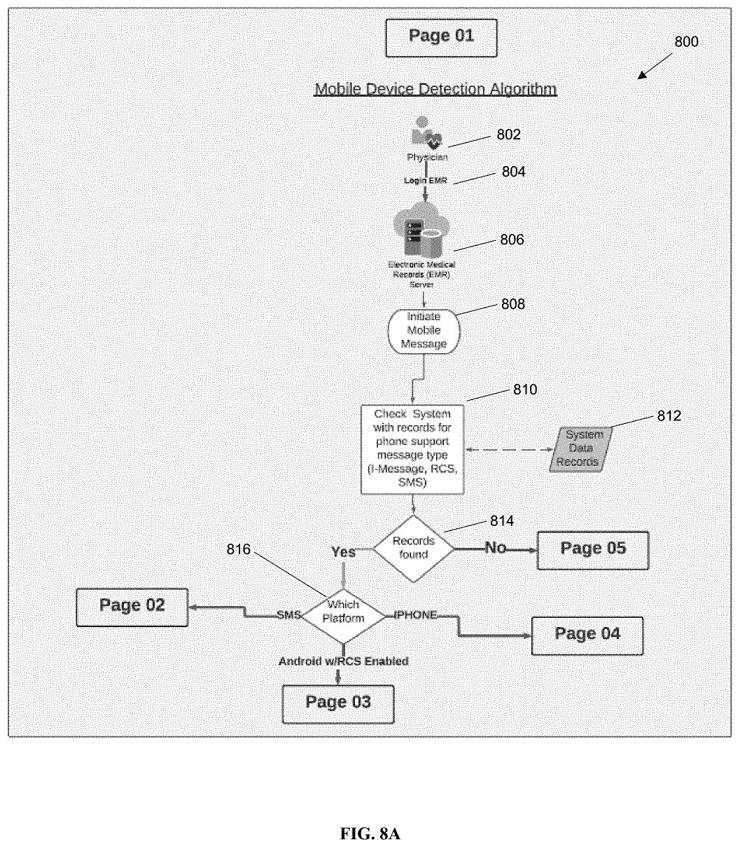

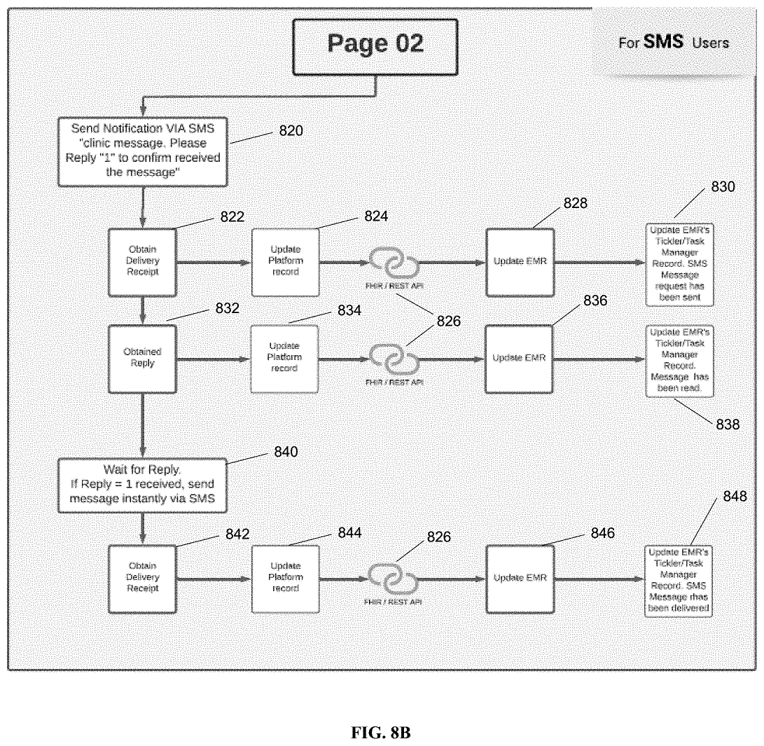

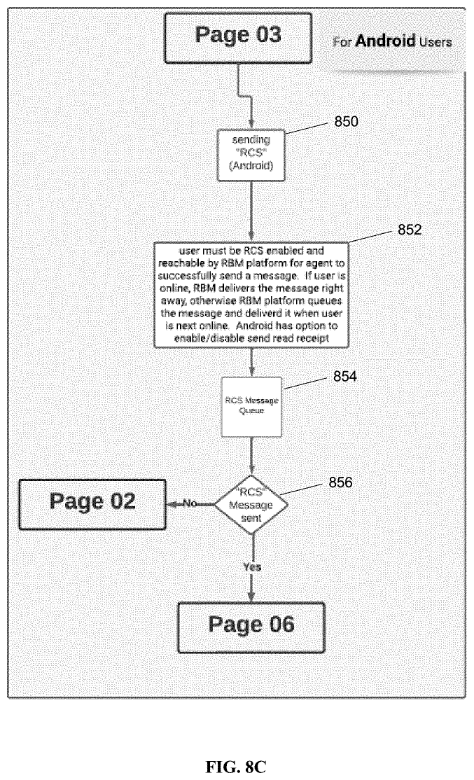

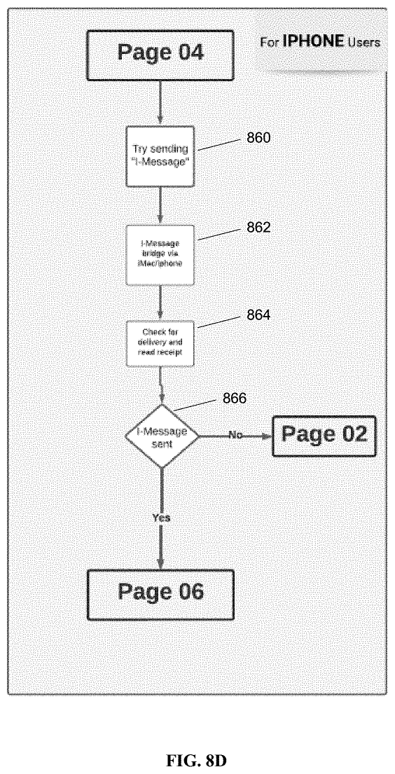

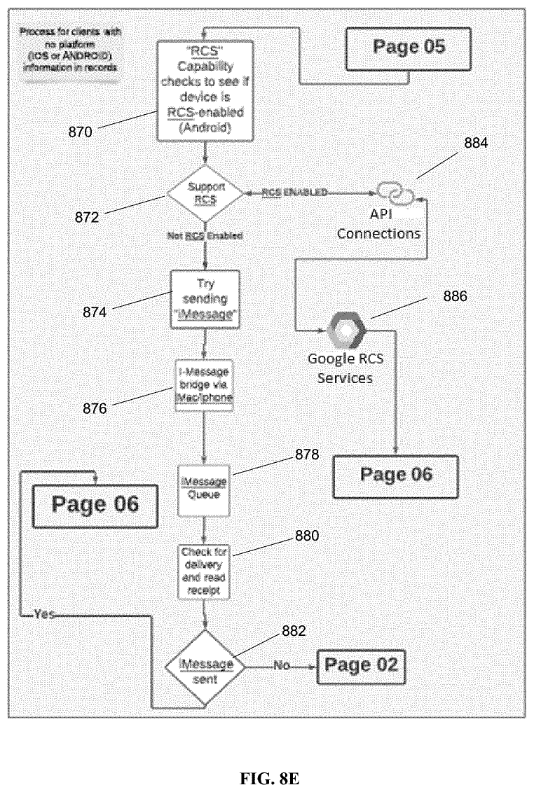

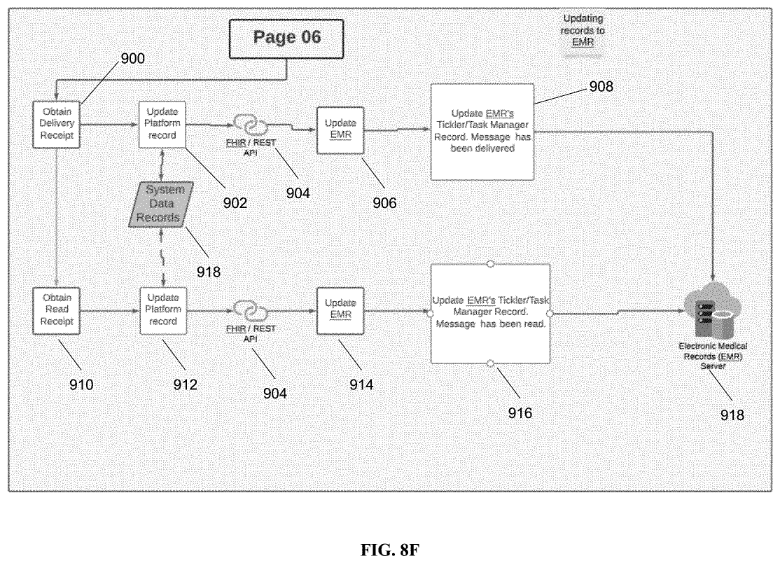

FIGS. 8A to 8F are diagrams illustrating an exemplary mobile device detection workflow. FIG. 8A is Page 01 of the mobile device detection workflow illustrating initiation steps. FIG. 8B is Page 02 of the mobile device detection workflow illustrating steps for SMS users. FIG. 8C is page 03 of the mobile device detection workflow illustrating steps for Android® users. FIG. 8D is page 04 of the mobile device detection workflow illustrating steps for iPhone® users. FIG. 8E is page 05 of the mobile device detection workflow illustrating steps for clients with no supported platform (i.e., not iOS or Android®). FIG. 8F is page 06 of the mobile device detection workflow illustrating steps for updating records to the EMR system.

According to FIG. 8A, EMR notification system 800 begins with a physician 802 (or clinic) logging into the EMR system at step 804 which will then connect to the EMR server 806. Next, the system would initiate a mobile message at step 808. The system will then check records for phone support message type (i.e., iMessage®, RCS or SMS) at step 810 by synching with system data records 812.

According to FIG. 8A, if no record is found, at step 814, the workflow will proceed to FIG. 8E (Page 05) for clients with no supported platform. If a record is found, the workflow moves to the next step to determine which platform at step 816. If the platform is SMS, then the workflow continues at FIG. 8B (Page 02). If the platform is Android® with RCS enabled, then the workflow continues FIG. 8C (Page 03). If the platform is iPhone® (i.e. Apple® iOS), the workflow continues at FIG. 8D (Page 04).

Referring to FIG. 8B (Page 02) of the mobile device detection workflow illustrating steps for SMS users, once the platform (at step 816) is determined to be SMS users, the system sends a notification via SMS at step 820. The message may contain such wording as “Clinic message. Please Reply 1 to confirm received message”. Next, the system obtains a delivery receipt at step 822 and updates the platform record at step 824 using a FIHR/REST API 826. The next step is to update the EMR record at step 828 and finally to update the EMR's tickler/task manager record at step 830 with a record that an SMS message request has been sent.

According to FIG. 8B, the next step is for the system to obtain a reply at step 832. Thereafter, the system updates the platform record at step 834 using a FIHR/REST API 826. The next step is to update the EMR record at step 836 and finally to update the EMR's tickler/task manager record at step 838 with a record that message has been read.

According to FIG. 8B, the next step is to wait for a reply at step 840. If the reply is received, a message is sent instantly via SMS. Thereafter, the system will obtain a delivery receipt at step 842 whereby the system will update the platform record at step 844 using a FIHR/REST API 826. The next step is to update the EMR at step 846 and update the EMR's tickler/task manager record at step 848 with a record that message has been delivered.

Referring to FIG. 8C (page 03), the workflow starts with selecting the Rich Communication Services (RCS) Android® as the communication protocol. The first step of FIG. 8C is sending an RCS message to the Android® device at step 850. Next, at step 852, the system determines whether RCS is enabled and reachable by the RBM platform for agent to successfully send a message. If the user is online, RBM delivers the message right away, otherwise the RBM platform queues the message and delivers it when the user is next online. Android® has an option to enable/disable the send receipt.

According to FIG. 8C, the RCS message is queued at step 854 and a decision is made on whether the “RCS” message is sent at step 856. If it is sent, then the workflow returns to step 820 of FIG. 8B (Page 02) for delivery for SMS users. If the message is not sent, the workflow moves to FIG. 8F (Page 06) to update records to EMR system.

Referring to FIG. 8D (page 04) for workflow for iPhone® users, the workflow starts with trying to send “iMessage®” at step 860. Next, the iMessage® bridges with the iMac®/iPhone® at step 862. Thereafter, the system checks for delivery and read receipt at step 864. Finally, the system determines whether the message is sent at step 866. If the message is sent, the workflow returns to step 820 of FIG. 8B (Page 02) for delivery for SMS users. If the iMessage® is not sent, the workflow moves to FIG. 8F (Page 06) to update records to EMR system.

Referring to FIG. 8E (page 05) for workflow for a process for clients with no platform (i.e., not iPhone® or iOS) records on file, the workflow starts with step 870 wherein the “RCS” capability checks to see if the device is RCS-enabled (for Android®). The next step is to check for support for RCS at step 872. If RCS not enabled, the workflow moves to attempt send the message as iMessage® at step 874. Next, the system bridges iMessage® to iMac®/iPhone® at step 876 and the iMessage® is queued at step 878.

According to FIG. 8E, the system then checks for delivery and read receipt at step 880 and then sends the iMessage® at step 882. If iMessage® is not sent, the workflow returns to FIG. 8B (Page 02), however if the message is sent, then the workflow continues onto FIG. 8F (Page 06) to update records to EMR system.

RCS is a protocol to send Android® messages to an Android® user. If the RCS is enabled, the system will try to send the message via Google® RCS services. Referring back to step 872 of FIG. 8E, if RCS is enabled, the system will use API Connections 884 to connect to Google® RCS Services 886 to send the message. Thereafter, the system moves to FIG. 8F (Page 06) to update records to EMR system.

Referring to FIG. 8F (Page 06) for workflow for updating records to EMR, the first step of the workflow is to obtain a delivery receipt at step 900. The next step is to update the platform record at step 902 using a FIHR/REST API at step 904. Next, the EMR is updated at step 906 and then the system updates the EMR's tickler/task manager record that the message has been delivered at step 908 where the data is finally saved in the EMR server 918.

According to FIG. 8F, after step 900, the workflow will obtain a read receipt at step 910 where it is sent to a mobile platform (i.e., iOS, Android®, SMS, etc.) to update the platform record at step 912 using a FIHR/REST API 904. Next, the EMR is updated at step 914 and then the system updates the EMR's tickler/task manager record that the message has been read at step 916 where the data is finally saved in the EMR server 918. Furthermore, both step 902 and 912 will check system data records at step 918.

According to further embodiments of this disclosure, messages created from the portal page can be sent as a text message. Furthermore, message can alternatively be sent as an email or can be texted to the landline to users without a mobile phone and only access to a landline. There, messages are also automatically updated in the patient's chart in an EMR system.

Further embodiments of this disclosure will provide techniques to send mobile message with delivery and read receipt update in EMR system as part of the EMR, and third party add on module.

Further embodiments of this disclosure will provide techniques to automate task with custom algorithm and logic to mark the task manager's task complete based on the read receipt status of the mobile message.

Further embodiments of this disclosure will provide techniques to support EMR system to send mobile message with delivery and read receipt update in EMR by direct lookup patent demographics record, phone type and phone number.

Further embodiments of this disclosure will provide techniques to support EMR system to send mobile message with w/delivery and read receipt update in EMR by integrating via EMR's task manager such as OSCAR Pro-Tickler.

Further embodiments of this disclosure will provide techniques to support Apple® iMessage®, Android® RCS, and SMS message with delivery & read receipt and update message record in EMR system.

Further embodiments of this disclosure will provide techniques for automated mobile message from the EMR system to the patient with delivery & read receipt confirmation.

Further embodiments of this disclosure will provide techniques to identify if phone is RCS Android® enable, iMessage® enable, or SMS enable.

Further embodiments of this disclosure will provide a system that can support other chat messenger not limited to Facebook® messenger, Whatsapp®, Google® Chat/Hangout, Signal®, and other chat messengers with delivery and read receipt and record in EMR system.

In further embodiments of this disclosure, further security enhancements can be added to the communication system including features related to security and authentication. In a further embodiment, two factor authentication may be implemented to authenticate the user to ensure that the intended user authenticates prior to receiving messages. For example, an SMS message may be sent to a phone for a user to receive a passcode (i.e., 6 digit password). The passcode will be delivered through another communication channel other than SMS (e.g., another phone number, home telephone line, email, etc.). Once the user receives the passcode and successfully enters it, the user can then download the message.

According to embodiments of the disclosure, a method of providing read and delivery receipts for mobile device messages from notifications from an EMR system is disclosed. The method comprises of the steps of logging into an online account of the EMR system, creating a data message to send to a mobile platform, selecting send message by the user, receiving the message at the EMR system, checking the system with records for mobile device support message type. If a record is not found, check to determine whether the mobile device is RCS-enabled Android® device and if RCS is not enabled, send iMessage® and check for delivery and read receipt.

According to the disclosure, if a record is found, determine a mobile device platform whereby if the platform is SMS, obtain delivery receipt, obtain a reply, update the EMR system. If the platform is Android®, send an RCS message if user is online, queue RCS message for further sending if user is offline, obtain the delivery receipt, obtain a read receipt, update the EMR system. Furthermore, if the platform is iOS for iPhone® devices, then send the iMessage®, check for delivery and read receipt, obtain delivery receipt, obtain a read receipt and update the EMR system.

According to the disclosure, the aforementioned method will conclude with the following steps: sending the message to the recipient, receiving the message on the recipient's mobile device and receiving notification of read and delivery receipt of the message at the user device. The method comprises a further step of selecting an option to provide read receipt to the recipient. The method further comprising the step of creating an account on EMR system if no account exists.

According to the disclosure, the mobile platform of the aforementioned method is selected from a list consisting of Android®, RCS, iOS, SMS, and email. The message type of the aforementioned method is selected from a list consisting of iMessage®, RCS, SMS and email. The communication with the EMR system is conducted using FIHR and REST APIs.

According to the disclosure, the aforementioned method further comprising the step of checking the system with records further comprising checking the system data records. The step of updating EMR system further comprising updating records that “Message has been sent”, “Message has been read” and “Message has been delivered”. The step of logging into an online account of the EMR system further comprises using two factor authentication. The two factor authentication further comprising receiving an SMS message passcode to the user mobile phone.

According to the disclosure, the aforementioned method further comprising translating the text message to another language using a translation service prior to delivery of message to the recipient.

According to the disclosure, an EMR notification system for providing read and delivery receipts for mobile device messages from notifications from an EMR system is also disclosed. The EMR notification system comprises a user computer to create a message, an EMR server in communication with EMR system, a database to store records, an EMR Connectivity module, a messenger module and a recipient mobile device with one or more supported platforms. The EMR notification system, in communication with the EMR system is configured to send the message to the recipient, receive the message on the recipient's mobile device, receive notification of read and delivery receipt of the message and update records of the EMR system with read and delivery receipt status.

According to the disclosure, the database of the EMR system is configured to store demographic information and further consists of a tickler and task system module. The EMR system further comprises a plurality of modules selected from a list consisting of an AllergyIntolerance module, an Appointment module, a Condition module, a DiagnosticReport module, a DocumentReference module, an Immunization module, an Invoice module, a Medication module, an Observation module, a Patient module, a Practitioner module, a Schedule Module and a Task module. Furthermore, the EMR system further comprises a translation module configured to translate the text messages to another language prior to delivery of the message to the recipient.

According to the disclosure, the EMR connectivity module of the EMR system is selected from list consisting of Task Manager, Patient lookup module, mobile message module, and translation module. The messenger module of the EMR system is selected form list consisting of iMessage® module, RCS Android® module and SMS module. The mobile platform of the EMR system is selected from list consisting of RCS Android®, iOS and SMS.

According to the disclosure, communication with the EMR system is conducted using FIHR and REST APIs. The step of updating records of EMR system further comprising updating records that “Message has been sent”, “Message has been read” and “Message has been delivered”. The EMR system further comprising supporting two factor authentication wherein two factor authentication consists of receiving an SMS message passcode to the user's mobile phone.

Implementations disclosed herein provide systems, methods and apparatus for generating or augmenting training data sets for machine learning training. The functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor. A “module” can be considered as a processor executing computer-readable code.

A processor as described herein can be a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be a controller, or microcontroller, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, any of the signal processing algorithms described herein may be implemented in analog circuitry. In some embodiments, a processor can be a graphics processing unit (GPU). The parallel processing capabilities of GPUs can reduce the amount of time for training and using neural networks (and other machine learning models) compared to central processing units (CPUs). In some embodiments, a processor can be an ASIC including dedicated machine learning circuitry custom-build for one or both of model training and model inference.

The disclosed or illustrated tasks can be distributed across multiple processors or computing devices of a computer system, including computing devices that are geographically distributed. The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.” While the foregoing written description of the system enables one of ordinary skill to make and use what is considered presently to be the best mode thereof, those of ordinary skill will understand and appreciate the existence of variations, combinations, and equivalents of the specific embodiment, method, and examples herein. The system should therefore not be limited by the above described embodiment, method, and examples, but by all embodiments and methods within the scope and spirit of the system. Thus, the present disclosure is not intended to be limited to the implementations shown herein but is to be accorded the widest scope consistent with the principles and novel features disclosed herein.

What is claimed:

1. A method of providing read and delivery receipts for mobile device messages from notifications from an EMR system, the method comprising:
logging into an online account of the EMR system;
creating a data message to send to a mobile platform;
selecting send message by the user;
receiving the message at the EMR system;
checking system with records for mobile device support message type;
if a record is not found, check to determine whether the mobile device is RCS-enabled Android® device;
if RCS is not enabled, send iMessage®;
check for delivery and read receipt;
if a record is found, determine a mobile device platform;
if the platform is SMS,
obtain delivery receipt;
obtain reply;
update EMR system;
if the platform is Android®,
send RCS message if user is online;
queue RCS message for further sending if user if offline;
obtain delivery receipt;
obtain read receipt;
update EMR system;
if the platform is iOS for iPhone® devices;
send iMessage®;
check for delivery and read receipt
obtain delivery receipt;
obtain read receipt;
update EMR system;
sending the message to the recipient;
receiving the message on the recipient mobile device; and
receiving notification of read and delivery receipt of the message at the user device.

2. The method of claim 1 further comprising the step of selecting an option to provide read receipt to the recipient.

3. The method of claim 1 further comprising the step of creating an account on EMR system if no account exists.

4. The method of claim 1 wherein the mobile platform selected from a list consisting of Android®, RCS, iOS, SMS, and email.

5. The method of claim 1 where the message type is selected from a list consisting of iMessage®, RCS, SMS and email.

6. The method of claim 1 further comprising the step of checking the system with records further comprising checking the system data records.

7. The method of claim 1 wherein communication with the EMR system is conducted using FIHR and REST APIs.

8. The method of claim 1 wherein the step of updating EMR system further comprising updating records that “Message has been sent”, “Message has been read” and “Message has been delivered”.

9. The method of claim 1 wherein the step of logging into an online account of the EMR system further comprises using two factor authentication.

10. The method of claim 9 wherein two factor authentication further comprising receiving a SMS message passcode to the user mobile phone.

11. The method of claim 1 further comprising translating the text message to another language using a translation service prior to delivery of message to the recipient.

12. An EMR notification system for providing read and delivery receipts for mobile device messages from notifications from an EMR system, the system comprising:
a user computer to create a message;
an EMR server in communication with EMR system;
a database to store records;
an EMR Connectivity module;
a messenger module; and
a recipient mobile device with one or more supported platforms;
wherein the EMR notification system, in communication with the EMR system is configured to:
send the message to the recipient;
receive the message on the recipient mobile device;
receive notification of read and delivery receipt of the message; and
update records of the EMR system with read and delivery receipt status.

13. The EMR system of claim 12 wherein the database is configured to store demographics info and further consists of a tickler and task system module.

14. The EMR system of claim 12 further comprising a plurality of modules selected from a list consisting of an AllergyIntolerance module, an Appointment module, a Condition module, a DiagnosticReport module, a DocumentReference module, an Immunization module, an Invoice module, a Medication module, an Observation module, a Patient module, a Practitioner module, a Schedule Module and a Task module.

15. The EMR system of claim 12 wherein the EMR connectivity module is selected from list consisting of Task Manager, Patient lookup module, mobile message module, and translation module.

16. The EMR system of claim 12 wherein the messenger module is selected form list consisting of iMessage® module, RCS Android® module and SMS module.

17. The EMR system of claim 12 wherein communication with the EMR system is conducted using FIHR and REST APIs.

18. The EMR system of claim 12 wherein the step of updating records of EMR system further comprising updating records that “Message has been sent”, “Message has been read” and “Message has been delivered”.

19. The EMR system of claim 12 further comprising supporting two factor authentication configured to receive a SMS message passcode to the user mobile phone.

20. The EMR system of claim 12 further comprising a translation module configured to translate the text messages to another language prior to delivery of the message to the recipient.
